(19) United States
(12) Patent Application Publication
(10) Pub. No.: US 2017/0287504 A1
(43) Pub. Date: Oct. 5, 2017

(54) APPARATUS TO ASSIST SPEECH TRAINING AND/OR HEARING TRAINING AFTER A COCHLEAR IMPLANTATION

(71) Applicants: Fraunhofer-Gesellschaft zur Förderung der angewandten Forschung e.V., Munich (DE); Technische Universität Dresden, Dresden (DE)

(72) Inventors: Uwe Vogel, Dresden (DE); Thomas Zahnert, Dresden (DE)

(21) Appl. No.: 15/473,025

(22) Filed: Mar. 29, 2017

(30) Foreign Application Priority Data
Apr. 1, 2016 (DE) 10 2016 105 970.6

(51) Int. Cl. G10L 25/48; A61N 1/36; G02B 27/01; G10L 15/26; G10L 21/10

(52) U.S. Cl. CPC G10L 25/48; G10L 15/26; G10L 21/10; G02B 27/017; A61N 1/36032; G02B 2027/0178; G02B 2027/0141

(57) ABSTRACT

An apparatus to assist speech training and/or hearing training after a cochlear implantation may include at least one first processor for converting a first signal containing speech information into a visual signal containing the speech information, and a display element with which the text of the speech information can be imaged after accessing of the visual signal. The apparatus may further include a cochlear module comprising a microphone for recording an acoustic signal containing speech information; at least one second processor for converting the acoustic signal containing the speech information into a digital signal containing the speech information, and a first transmitter for transmitting the digital signal containing the speech information; and a first receiver for receiving the digital signal containing the speech information, which can be delivered to the first processor as a first signal.

### CROSS-REFERENCE TO RELATED APPLICATIONS

This application claims priority under 35 USC § 119 to German patent application 10 2016 105 970.6 filed on Apr. 1, 2016, entitled “Vorrichtung zum Unterstützen des Sprach-und/oder Hörtrainings nach einer Cochlear Implantation.”

### BRIEF DESCRIPTION OF THE DRAWINGS

FIG. 1 is a schematic representation of components of an apparatus according to the invention; and FIG. 2 depicts parts of the individual components in a block diagram.

### DETAILED DESCRIPTION

The invention relates to an apparatus to assist speech training and/or hearing training after a cochlear implantation by visual feedback of speech to a rehabilitating patient. The apparatus according to the invention is furthermore distinguished in that it can be used in the everyday environment of a user.

In Germany alone, about 3000 to 4000 patients are treated with a cochlear implant (also referred to by the abbreviation CI below) each year. A CI (DE 10 2005 049 507 A1) essentially consists of two modules, a module referred to below as the cochlear module, which is arranged externally on the skin of a patient's head, and a module, the actual implant, which is fitted surgically under the skin of the head.

A cochlear module conventionally comprises a microphone, a microprocessor and a transmitter coil. The microphone is used to record an acoustic signal, which is delivered to the microprocessor and is converted by means of the microprocessor into a multichannel digital signal. By means of the transmitter coil, the multichannel digital signal is transmitted through the skin of the head to a receiver of the implant, and is forwarded from the receiver to a multichannel electrode. The pulse-shaped signals conducted into the multichannel electrode stimulate the nerve structures of the cochlea. In this way, the cochlear nerve receives the signal and conducts it to the auditory cortex in the brain.

With the advent of CI operations, initially above all children born deaf were treated, for whom hearing was actually made possible for the first time in this way. Since then, about 60% of CI treatments have been given to patients who cannot achieve sufficient speech understanding with conventional hearing aids because of their great hardness of hearing in old age. This also includes patients born hard of hearing, who likewise no longer benefit from conventional hearing aids because of the progression of their disease. On the basis of the peripheral as well as central deficiencies in sound/speech processing experienced with hardness of hearing, patients require a month or year of rehabilitation in speech perception after cochlear implantation. This rehabilitation currently takes place in repeated static training, during which patients receive visual feedback of speech. A disadvantage with this is that a patient can only carry out this training in treatment centres provided therefor.

Yet other apparatuses and methods incorporating modern technology have now been disclosed, by means of which speech and/or hearing can be trained after a cochlear implantation.

For example, Android-based application software (also referred to below as an app) with the name “Hearing Training for CIs” is known from the German Hearing Centre Hanover, by means of which hearing can be trained after a cochlear implantation. In this case, for CI patients, an android device, for example a smartphone or tablet PC, is connected by means of a suitable and approved cable and consonant tests or one-, two- or three-syllable word tests are played by means of the app installed on a smartphone. In this case, it is possible to adjust a threshold of hearing as a minimum volume, as well as a maximum volume used by the program. A disadvantage with this is that speaking cannot be trained by means of such an apparatus.

A further Android app named “Captioning on Glass” has been developed by the Georgia Tech Research Corporation for communication between two persons, at least one of whom may have impaired hearing. By means of this app, an Android-based smartphone and a spectacle display known by the term “Google Glass” (U.S. Pat. No. 9,195,067 B1) are combined with one another. By means of the microphone of the smartphone, the sound waves of the speech of one person are received and the received acoustic signal is converted by means of a processor of the smartphone into a signal with which the spoken text can be visually imaged. This signal is then transmitted to the spectacles by the smartphone, and with the aid of the spectacles the text embodied in the signal is projected into the field of view of the second person, who wears the spectacles. A disadvantage with this is that the microphone must be held very close during speaking in front of the mouth of a person speaking, in order to pick up as little background noise as possible, for which reason this apparatus is suitable only for a limited number of everyday situations.

The technical object of the invention is to provide an apparatus for speech training and/or hearing training after a cochlear implantation, by means of which the disadvantages of the prior art can be overcome. In particular, the apparatus is intended to shorten the total rehabilitation time of a patient after a CI implantation, while reducing the staff outlay and above all being usable in the everyday environment of the patient.

An apparatus according to the invention to assist speech training and/or hearing training after a cochlear implantation comprises at least one first processor for converting a first signal containing speech information into a visual signal containing the speech information, and a display element with which the text of the speech information can be imaged after accessing of the visual signal.

A further part of an apparatus according to the invention is a cochlear module. This comprises a microphone for recording an acoustic signal containing speech information; at least one second processor for converting the acoustic signal containing the speech information into a digital signal containing the speech information, and a first transmitter for transmitting the digital signal containing the speech information. The first transmitter may in this case comprise the transmission coil by means of which the digital signals generated by the processor of a cochlear module are transmitted to the receiver of a cochlear implant underneath the skin of the head. As an alternative, the first transmitter may also be configured as a separate additional transmitter.

An apparatus according to the invention furthermore has a first receiver for receiving the digital signal containing the speech information, which can be delivered to the first processor as a first signal.

By means of an apparatus according to the invention, an acoustic speech signal, which is recorded with the microphone of a cochlear implant, is fed back visually as text to the person wearing the cochlear implant, so that it is possible to train hearing of their own speech and the speech of other persons, as well as the actual speech of a CI patient.

In one embodiment, the first receiver, the first processor and the display element are parts of the same device. Such a device may, for example, be configured as a smartphone or tablet PC. For example, the digital signals containing speech information are transmitted by the transmitter of the cochlear module by means of Bluetooth, Wi-Fi, or any other wireless transmission standard to the smartphone, or the tablet PC, converted into a visual signal in a processor of the smartphone or of the tablet PC, and imaged as text on the display of the smartphone or of the tablet PC. Application software, by means of which an audio speech signal can be converted into a visual text signal, is known and can be installed on a smartphone or a tablet PC. The transmitter of the cochlear module may be any wireless or wired transmitter of electromagnetic signals. In some examples, the transmitter may be part of a transceiver. Correspondingly, the first receiver may be any wireless or wired receiver of electromagnetic signals, such as a Bluetooth or Wi-Fi receiver. In some examples, the first receiver may be part of a transceiver.

In an alternative embodiment, the first receiver and the first processor are parts of a first device, and the display element is part of a second device. In this case, the first device comprises a second transmitter for transmitting the visual signal and the second device comprises a second receiver for receiving the visual signal. The first device may, for example, be configured as a smartphone or tablet PC, and the second device may comprise a projector or a display not belonging to the smartphone or tablet PC. The display may be configured as a spectacle display, such as is known by the name “Google Glass”, for example. The second transmitter may be any wireless or wired transmitter of electromagnetic signals. Correspondingly, the second receiver may be any wireless or wired receiver of electromagnetic signals, such as a Bluetooth or Wi-Fi receiver. In some examples, the second transmitter and/or the second receiver may be part of a transceiver.

In one embodiment of the invention, the second device configured as a spectacle display has at least one optical element, by means of which the text imaged on the spectacle display can be projected on a magnified scale into the field of view of a person wearing the spectacle display.

It should be noted that the term spectacle display used in the description of the invention is not restricted only to a spectacle display known as “Google Glass”, but rather includes all known display elements which can be arranged in the vicinity of a person's eye and with which an image, video or text can be overlaid or projected into the person's field of view.

The signal transmission from the first transmitter to the first receiver and/or from the second transmitter to the second receiver may, for example, be carried out by means of the Bluetooth or Wi-Fi standard, in which case an app, with which the signal exchange between the cochlear module, the first device and/or the second device, as well as the conversion of signals involved in the signal exchange, can be controlled, may be stored in a processor of the first device.

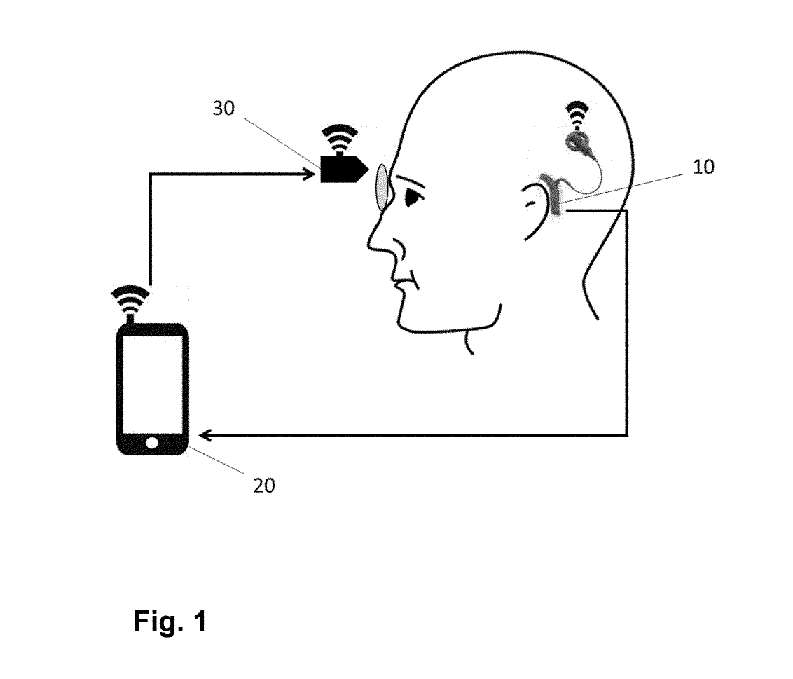

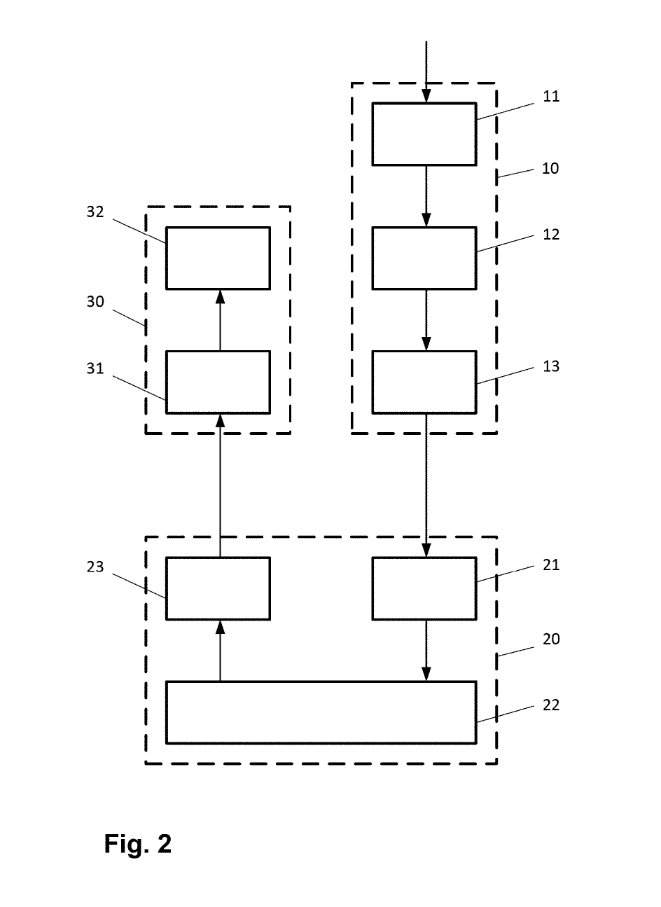

The invention will be explained in more detail below with the aid of a preferred exemplary embodiment. FIGS. 1 and 2 illustrate an apparatus or a system according to the invention, FIG. 1 schematically representing only the components of an apparatus according to the invention schematically, and FIG. 2 also depicting some parts of the individual components in a block diagram.

An apparatus according to the invention, corresponding to the preferred exemplary embodiment, comprises three separate components, a cochlear module 10, a first device 20 configured as a smartphone or tablet PC, and a second device 30 configured as a spectacle display.

By means of a microphone 11 of the cochlear module 10, an acoustic signal containing speech information is recorded and forwarded to a processor 12. The processor 12 converts the acoustic signal into a digital signal containing the speech information, which is transmitted by means of a first transmitter 13 to a first receiver 21 of the first device 20. The first receiver 21 delivers the digital signal to a processor 22, by means of which the digital signal is converted into a visual signal containing the speech information. The visual signal containing the speech information is subsequently transmitted by means of a second transmitter 23 of the first device 20 to a second receiver 31 of the second device 30, where it is delivered to a display element 32 configured as a display, on which the text of the speech information is imaged. The second device 30 configured as a spectacle display furthermore comprises an optical element (not represented in FIGS. 1 and 2), by means of which the text imaged on the display element 32 is projected on an enlarged scale into the field of view of a person wearing the spectacle display. Such optical elements are conventional parts of known spectacle displays.

During use of an apparatus according to the invention, the cochlear module 10 is usually fastened in the vicinity of an ear by means of a magnet, which sticks to the implant lying below the skin of the head. A first device 20 configured as a smartphone may be carried inside a pocket of a patient's clothing, and a second device 30 configured as a spectacle display may be worn as spectacles. The hands of a CI patient therefore remain free, so that an apparatus according to the invention is suitable for everyday life. In this way, a faster learning outcome in the CI rehabilitation phase and a reduction of the total rehabilitation time for a patient are possible. A CI rehabilitant is no longer dependent on carrying out the speech and/or hearing training in a special training centre, so that the staff outlay, the costs of rehabilitation and the travel expenses for a patient can be reduced.

Examples of the processors 12 and 22 may include a general processor, a central processing unit, a microcontroller, an application specific integrated circuit (ASIC), a digital signal processor, a field programmable gate array (FPGA), a digital circuit, and an analog circuit. Any software described herein, such as the software application and/or the app described herein may be stored in memory (not shown). The memory may be any device for storing and retrieving data or any combination thereof. The memory may include non-volatile and/or volatile memory, such as a random access memory (RAM), a read-only memory (ROM), an erasable programmable read-only memory (EPROM), or flash memory. Alternatively or in addition, the memory may include an optical, magnetic (hard-drive) or any other form of data storage device. The software may include code executable by a processor, such as the processor 12 configured to convert the acoustic signal into the digital signal containing the speech information and/or the processor 22 that is configured to convert the digital signal into the visual signal containing the speech information.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

1. An apparatus to assist speech training and/or hearing training after a cochlear implantation, the apparatus comprising:
a first processor configured to convert a first signal that includes speech information into a visual signal that includes the speech information;
a display element configured to display text of the speech information from the visual signal;
a cochlear module comprising a microphone configured to record an acoustic signal that includes the speech information;
a second processor configured to convert the acoustic signal that includes the speech information into a digital signal that includes the speech information;
a first transmitter configured to transmit the digital signal that includes the speech information; and
a first receiver configured to receive the digital signal that includes the speech information, which is deliverable to the first processor as the first signal.

2. The apparatus of claim 1, wherein the first receiver, the first processor and the display element are parts of one device.

3. The apparatus of claim 2, wherein the one device is configured as a smartphone or as a tablet PC.

4. The apparatus of claim 1, wherein the first receiver and the first processor are parts of a first device, and the display element is part of a second device, the first device comprising a second transmitter configured to transmit the visual signal and the second device comprising a second receiver configured to receive the visual signal.

5. The apparatus of claim 4, wherein the first device is configured as a smartphone or as a tablet PC.

6. The apparatus of claim 5, wherein the second device comprises a projector.

7. The apparatus of claim 5, wherein the second device comprises a display.

8. The apparatus of claim 4, wherein the second device comprises a projector.

9. The apparatus of claim 4, wherein the second device comprises a display.

10. The apparatus of claim 9, wherein the second device is configured as a spectacle display.

11. The apparatus of claim 10, wherein the spectacle display has at least one optical element, by means of which the text imaged on the spectacle display can be projected on a magnified scale into the field of view of a person wearing the spectacle display.

12. The apparatus of claim 4, wherein an app, with which the signal exchange between the cochlear module, the first device and/or the second device, as well as the conversion of signals involved in the signal exchange, can be controlled, is stored in a processor of the first device.

13. The apparatus of claim 1, wherein the first transmitter and the first receiver and/or the second transmitter and the second receiver are suitable for signal transmission by means of the Bluetooth or Wi-Fi standard.
